(19) United States
(12) Patent Application Publication
(10) Pub. No.: US 2021/0341488 A1
(43) Pub. Date: Nov. 4, 2021

(54) FLUORESCENCE POLARIZATION IMMUNOASSAY AND FLUORESCENT LABELING SUBSTANCE

(71) Applicants: TOHOKU UNIVERSITY, Sendai-shi (JP); Tianma Japan, Ltd., Kawasaki (JP); NATIONAL UNIVERSITY CORPORATION HOKKAIDO UNIVERSITY, Sapporo-shi (JP)

(72) Inventors: Mao Fukuyama, Sendai-shi (JP); Akihide Hibara, Sendai-shi (JP); Ayuko Imai, Kawasaki (JP); Koji Shigemura, Kawasaki (JP); Manabu Tokeshi, Sapporo-shi (JP)

(73) Assignees: TOHOKU UNIVERSITY, Sendai-shi (JP); Tianma Japan, Ltd., Kawasaki (JP); NATIONAL UNIVERSITY CORPORATION HOKKAIDO UNIVERSITY, Sapporo-shi (JP)

(21) Appl. No.: 17/241,861

(22) Filed: Apr. 27, 2021

(30) Foreign Application Priority Data
Apr. 30, 2020 (JP) 2020-080767

(51) Int. Cl. G01N 33/58; G01N 21/64

(52) U.S. Cl. CPC G01N 33/582; G01N 2201/062; G01N 2021/6439; G01N 21/6428

(57) ABSTRACT

A fluorescence polarization immunoassay that uses a fluorescent labeling substance in which a single domain antibody is labeled with a fluorescent dye. A fluorescent labeling substance obtained by labeling a single domain antibody, such as a VHH antibody or a vNAR antibody, having a binding ability to a target substance with a fluorescent dye is used. The fluorescence polarization immunoassay includes a binding step for binding the fluorescent labeling substance to a target substance; and a measuring step for measuring a change in the fluorescence polarization of the fluorescent labeling substance to which the target substance is bound.

### CROSS-REFERENCE TO RELATED APPLICATION

This application claims the benefit of Japanese Patent Application No. 2020-80767, filed on Apr. 30, 2020, the entire disclosure of which is incorporated by reference herein.

### FIELD

The present disclosure relates to a fluorescence polarization immunoassay, using a fluorescent labeling substance in which a fluorescent dye is bound to a single domain antibody, and the fluorescent labeling substance.

### BACKGROUND

Fluorescence polarization immunoassay is known as an immunity analysis method using fluorescence. As described in X. Q. Guo et al., Anal. Chem. 1998, 70, 632-637, there is the following relationship between the fluorescence polarization and the volume of the substance being measured. (1/P−⅓)=(1/P₀−⅓)(1+kTτ/ηV) P: fluorescence polarization, P₀: polarization observed in the absence of rotational diffusion, k: Boltzmann's constant, η: viscosity of the solution, T: absolute temperature in kelvin, τ: fluorescence lifetime, V: molecular volume.

Unexamined Japanese Patent Application Publication No. H03-103765 describes a fluorescence polarization immunoassay which uses a reagent in which an antibody (or antigen) is immobilized on a substance having a larger molecular weight than the antibody, then, utilizes a significant change in the fluorescence polarization caused by a specific antigen-antibody reaction between this reagent and a fluorescently labeled antigen (or antibody).

There is a method by measuring a high molecular weight substance using a fluorescence polarization immunoassay also (Japanese Patent No. 3255293). This method is characterized by the use of a fluorescently labeled protein in which a fluorescent dye having the fluorescence life of 10 to 200 nanoseconds is covalently bound to an antibody or the like that specifically binds to a target substance. In an example of the method, pyrenebutanoic acid that is a long-life fluorescent dye is used as the fluorescent dye, and a high density lipoprotein (HDL) calibration curve is created using an anti-HDL polyclonal antibody as the antibody that specifically binds to a target substance, as well as, a low density lipoprotein (LDL) calibration curve is created using an anti-LDL polyclonal antibody as the antibody that specifically binds to a target substance.

Further, another method is known for measuring a high molecular weight substance, which uses a fluorescent labeling substance obtained by fluorescently labeling a low molecular weight antibody such that a change in molecular weight before and after binding between the fluorescent labeling substance and a target substance becomes more significant (Unexamined Japanese Patent Application Publication No. H11-44688). Unexamined Japanese Patent Application Publication No. H11-44688 lists, as the low molecular weight antibody, a Fab fragment, a Fab′ fragment, and an scFv antibody (a single chain antibody) that contain at least an antigen recognition site. In an example thereof, a Fab-labeled compound is prepared by reacting a Fab fragment with fluorescein isothiocyanate (FITC), and various concentrations of human serum albumin are added to the Fab-labeled compound to measure fluorescence polarization. Unexamined Japanese Patent Application Publication No. H11-44688 discloses, as a result, that a low concentration region can be measured up to about 2 to 3×10⁻⁸ M (mol/L) (1 to 2 μg/mL).

Antibodies such as IgG antibodies are Y-shaped antibodies each comprising two heavy chains and two light chains, and each heavy chain and each light chain have a variable region. Fab antibodies and scFv antibodies comprise a part of such antibodies and contain a variable region of a heavy chain and a variable region of a light chain. Whereas, there are also heavy chain antibodies that each have two heavy chains bound in a Y shape without containing a light chain. Each heavy chain of the heavy chain antibody has a variable region. The variable region includes a framework region consisting of F1 to F4 and a complementarity determining region (CDR) consisting of CDR1 to CDR3 and such a variable region enables specific binging to an antigen. Since the variable region exerts antigen-binding properties by the framework region and CDR, if the variable region is defined as a single unit of domain (hereinafter, referred to as a single domain), a heavy chain antibody has two single domains. Heavy chain antibodies are contained in the serum of camelids, and each variable region of the heavy chain is called the variable domain of a heavy chain antibody (VHH). A camel-derived VHH antibody is a single domain antibody. Unexamined Japanese Patent Application Publication No. 2019-210267 describes a thermostable VHH antibody in which a specific amino acid of the VHH antibody is replaced with a glycine or the like for the purpose of thermostabilizing the VHH antibody.

The fluorescence polarization immunoassay is used to observe a change in the fluorescence polarization of a fluorescent labeling substance before and after binding to a target substance. Since the fluorescence polarization depends on the molecular weight, it is not easy to measure a high molecular weight substance using a fluorescent labeling substance having a large molecular weight. Japanese Patent No. 3255293 uses pyrenebutanoic acid that is a long-life fluorescent dye to prepare a fluorescent labeling substance and uses the fluorescent labeling substance for measuring a high molecular weight target substance (about 500,000 or more), for example, a target substance of a size larger than a virus (about 20 nm or more as a particle). However, long-life fluorescent dyes are expensive, thus, it is desired to develop a fluorescence polarization immunoassay that is capable of measuring a high molecular weight substance using a highly versatile fluorescent dye such as fluorescein with the fluorescence life of 10 nanoseconds or less.

Further, Unexamined Japanese Patent Application Publication No. H11-44688 suggests measurement possibility of up to about 2 to 3×10⁻⁸ M (mol/L) using a low molecular antibody. However, it is desired to develop a fluorescence polarization immunoassay that allows detection even at lower concentration in order to enable measurement with a trace amount of sample.

Furthermore, although Unexamined Japanese Patent Application Publication No. 2019-210267 discloses a VHH antibody having high thermal stability, the disclosure is about preparation of a variant and provides no example of using the VHH antibody for a fluorescence polarization immunoassay. Nor does the disclosure include description about other single domain antibodies than the VHH antibody.

Furthermore, in order to enable measurement with a trace amount of sample, a measuring instrument equipped with a microchannel has been developed. The microchannel is a device having a channel of about several ten to several hundred micrometers that is manufactured using a semiconductor process. A chemical reaction system using this microchannel has an advantage of enabling a stable chemical reaction due to significantly shortened reaction time and excellent uniformity of temperature and concentration as compared with a usual bulk size chemical reaction. The equipment used in a fluorescence polarization immunoassay must be made of a material that does not affect the fluorescence polarization, and, for this reason, polydimethylsiloxane (PDMS) is often used. PDMS has transparency comparable to quartz glass and has low autofluorescence, so PDMS is suitable for analysis that utilizes fluorescence reactions. In addition, since PDMS has low viscosity as liquid, the use of PDMS enables microfabrication on the order of submicrons to microns. However, the fluorescence polarization immunoassay utilizes antigen-antibody reactions. If a fluorescent labeling substance, a target substance, or a conjugate thereof adheres to PDMS, accurate measurement cannot be performed.

### SUMMARY

A fluorescence polarization immunoassay is for analyzing a target substance contained in a sample and the fluorescence polarization immunoassay comprises:

a binding step for binding a fluorescent labeling substance, in which a single domain antibody having a binding ability to the target substance is labeled with a fluorescent dye, to the target substance contained in the sample; and

a measuring step for measuring a change in fluorescence polarization of the fluorescent labeling substance to which the target substance is bound.

The fluorescent labeling substance is a fluorescent labeling substance in which a fluorescent dye is bound to a single domain antibody.

It is to be understood that both the foregoing general description and the following detailed description are exemplary and explanatory and are not restrictive of this disclosure.

### BRIEF DESCRIPTION OF THE DRAWINGS

A more complete understanding of this application can be obtained when the following detailed description is considered in conjunction with the following drawings, in which:

FIG. 1 is a diagram explaining a relationship among the mass a target substance, fluorescence polarization, and the fluorescence life of a fluorescent dye;

FIG. 2 is a diagram illustrating the result of a standard curve of rabbit IgG by the fluorescence polarization measurement of Example 1, and the result of a standard curve of rabbit IgG by the fluorescence polarization measurement of Comparative Example 1;

FIG. 3 is a diagram according to Example 2 illustrating the result of a standard curve of Her2 using a fluorescent labeling substance in which Alexa Fluor 488 is bound to the SH group of an antibody;

FIG. 4 is a diagram according to Example 3 illustrating the result of a standard curve of Her2 using a fluorescent labeling substance in which Alexa Fluor 488 is bound to the amino group of an antibody;

FIG. 5 is a diagram illustrating a schematic structure of a fluorescence polarization measuring device used in Example 4;

FIG. 6 is a diagram explaining microchannels in an effective visual field for observing a sample illumination part;

FIG. 7 is a diagram illustrating an image of the measured fluorescence polarization of the microchannels used in Example 4;

FIG. 8 is a diagram illustrating a standard curve of rabbit IgG by the fluorescence polarization measurement of Example 4, and the result of the fluorescence polarization of a sample for measurement;

FIG. 9 is a diagram illustrating the result of a standard curve of rabbit IgG by the fluorescence polarization measurement of Example 5; and

FIG. 10 is a diagram illustrating the result of a standard curve of rabbit IgG by the fluorescence polarization measurement of Comparative Example 2.

### DETAILED DESCRIPTION

A first embodiment of the present disclosure is a fluorescence polarization immunoassay for analyzing a target substance contained in a sample and the fluorescence polarization immunoassay comprises:

a binding step for binding a fluorescent labeling substance, in which a single domain antibody having a binding ability to the target substance is labeled with a fluorescent dye, to the target substance contained in the sample; and

a measuring step for measuring a change in fluorescence polarization of the fluorescent labeling substance to which the target substance is bound. The detection sensitivity improves with the use of a single domain antibody, enabling measurement of a sample that contains a target substance at low concentration and measurement of a high molecular weight target substance.

It is known that heavy chain antibodies comprising only heavy chains are produced in the bodies of camelids such as Bactrian camels, dromedary camels, llamas, alpacas, vicunas, and guanacos, and cartilaginous fish such as sharks and rays. The variable region of a heavy chain antibody derived from a camelid is called a VHH antibody, and the variable region of a heavy chain antibody derived from a cartilaginous fish is called a vNAR (variable new antigen receptor) antibody. The variable region of each heavy chain of the heavy chain antibody is a single domain antibody that exerts antigen-binding properties by the framework region and CDR. The “single domain antibody” herein means an antibody composing by one variable region. VHH and vNAR antibodies can be used as single domain antibodies.

The single domain antibody used in the present disclosure is limited to those having a binding ability to a specific target substance. When at least a part of a target substance is defined as an epitope, the single domain antibody is required to have a binding ability capable of recognizing and binding to this epitope.

The single domain antibody having such a binding ability can be prepared by preparing a heavy chain antibody that reacts with a target substance as an antigen and cutting out a part of the heavy chain antibody by cleavage or the like. For example, a heavy chain antibody-producing animal can be immunized with a target substance as an antigen, and a heavy chain antibody that binds to the antigen can be selected from B cells of the immunized animal. A variable region of a VHH antibody, a vNAR antibody or the like obtained by cleaving the heavy chain antibody with an enzyme or the like can be used as the single domain antibody. Moreover, the single domain antibody is not limited to an isolate from a heavy chain antibody. The single domain antibody may be one that is produced by genetic engineering to have a specific binding ability to a specific substance by referring to the DNA sequences of conventionally known VHH antibodies and vNAR antibodies or by using an antibody library or the like. Furthermore, in the single domain antibody that was prepared in such a way, some amino acids may be replaced with other amino acid residues for the purpose of improving heat resistance, chemical resistance, pressure resistance, and the like to the extent that the binding property to the target substance is not impaired. Further, a conventionally known Fab antibody or scFv antibody may be decomposed to extract one variable region that may be used as the single domain antibody.

The single domain antibody used in the present disclosure is labeled with a fluorescent dye, which is then used as a fluorescent labeling substance.

The “fluorescence” herein means light emission generated when light that excites electrons is irradiated. Further, the “fluorescent dye” means a dye that emits fluorescence. The fluorescent dye herein also includes a dye that emits phosphorescence since phosphorescence is also emitted when an atom absorbs energy and becomes excited just as fluorescence. If a fluorescent dye emits phosphorescence, the fluorescence polarization may be measured based on phosphorescence instead of fluorescence.

Examples of the fluorescent dye that can be used in the present disclosure include fluorescein compounds such as chlorotriazinyl aminofluorescein, 4′-aminomethylfluorescein, 5-aminomethylfluorescein, 6-aminomethylfluorescein, 6-carboxyfluorescein, 5-carboxyfluorescein, 5- and 6-aminofluorescein, thioureafluorescein, and methoxytriazinylaminofluorescein; nitrobenzoxadiazole derivatives such as nitrobenzoxadiazole chloride; indolenine; dansyl derivatives such as dansyl; naphthalene derivatives such as dialkylaminonaphthalene and dialkylaminonaphthalenesulfonyl: pyrene derivatives such as N-(1-pyrenyl) maleimide, aminopyrene, pyrenebutanoic acid, and alkynylpyrene; metal complexes such as platinum, rhenium, ruthenium, osmium, and europium; rhodamine derivatives such as rhodamine B, rhodamine 6G, and rhodamine 6GP; and, as registered trademark or product names, Alexa Fluor series such as Alexa Fluor 488, BODIPY series, DY series, ATTO series, Dy Light series, Oyster series, HiLyte Fluor series, Pacific Blue, Marina Blue, Acridine, Edans, Coumarin, DANSYL, FAN, Oregon Green, Rhodamine Green-X, NBD-X, TET, JOE, Yakima Yellow, VIC, HEX, R6G, Cy3, TAMRA, Rhodamine Red-X, Redmond Red, ROX, Cal Red, Texas Red, LC Red 640, Cy5, Cy5.5, and LC Red 705. Ruthenium emits phosphorescence, and its fluorescence life is 2,700 nanoseconds.

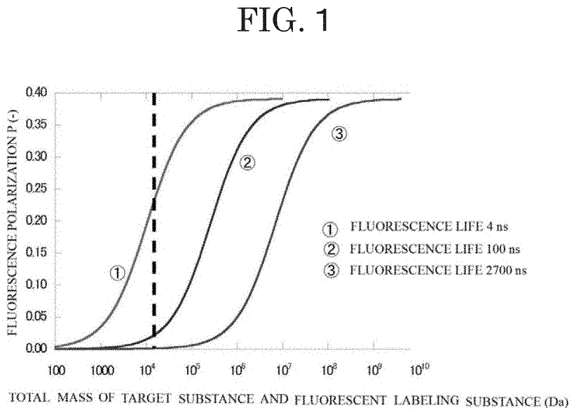

FIG. 1 schematically illustrates a relationship among the fluorescence life of a fluorescent dye, fluorescence polarization, and a molecular weight. This diagram was created with reference to “Use of a Long-Lifetime Re(I) Complex in Fluorescence Polarization Immunoassays of High-Molecular-Weight Analytes”, Analytical Chemistry, 1998, Vol. 70, P632. The horizontal axis of FIG. 1 represents molecular weight, and the vertical axis represents fluorescence polarization. FIG. 1 indicates that the total mass region that can be measured by fluorescence polarization differs depending on the fluorescence life of a fluorescent dye and that concentration of a target substance can be quantified when the fluorescence polarization is within a predetermined range (a range of about 0.05 to 0.35 in FIG. 1). For example, the fluorescence polarization changes significantly within a range of 1×10³ to 1×10⁵ (Da) when a fluorescent dye with the fluorescence life of four nanoseconds is used, within a range of 1×10⁴ to 1×10⁷ (Da) when a fluorescent dye with the fluorescence life of 100 nanoseconds is used, and within a range of 1×10⁶ to 1×10⁸ (Da) when a fluorescent dye with the fluorescence life of 2,700 nanoseconds is used. Fluorescence polarization is cancelled by rotational diffusion of a fluorescent labeling substance to which a target substance is bound between the time a fluorescent dye is excited and the time the fluorescent dye emits fluorescence. If a longer-life fluorescent dye is used, a change in the fluorescence polarization can be measured in a higher molecular weight region. As such, for example, when a fluorescent dye having the fluorescence life of four nanoseconds is bound to an IgG antibody of 150 kDa and used as a fluorescent labeling substance, the fluorescent labeling substance already has the fluorescence polarization of 0.37, and the fluorescence polarization remains almost unchanged even when the fluorescent labeling substance binds to a target substance with high molecular weight. In an example of Japanese Patent No. 3255293, high molecular weight substances such as C-reactive protein (CRP; molecular weight of 120,000), high-density lipoprotein (HDL; molecular weight of about 400,000) and low-density lipoprotein (LDL; molecular weight of 3 million) are measured by reducing the fluorescence polarization of a fluorescent labeling substance alone by using a long-life dye, such as a pyrene derivative, for an IgG antibody.

On the other hand, in the present disclosure, a fluorescent dye having the fluorescence life of 4 to 3,000 nanoseconds can be used regardless of the molecular weight of a target substance. However, a fluorescent dye may be appropriately selected within the above fluorescence life range according to measurement conditions, such as the molecular weight of a target substance and an excitation wavelength, and the autofluorescence of a sample for measurement. For example, when the mass of a target substance is about 15,000 to 2×10⁵ Da, a conventional fluorescent dye having the fluorescence life of about four nanoseconds may be used, and when the mass of a target substance is about 2×10⁵ to 2×10⁸ Da, a fluorescent dye having the fluorescence life of about 100 nanoseconds may be used. As will be described later in an example, in the present disclosure, a target substance having a high molecular weight of about 150 kDa can be quantified by using a fluorescent dye with a short fluorescence life, such as Alexa Fluor 488. Moreover, the lower limit of quantification is calculated to be 0.45 nM (nanomol/L), allowing detection at low concentration. Thus, although the reason is not clear why a highly sensitive and high molecular weight target substance can be measured using a highly versatile fluorescent dye such as Alexa Fluor 488, it is presumed that the sensitivity of the fluorescence polarization has increased by synergistical effects of factors, such as the single domain antibody having a low molecular weight, as well as, a fluorescent dye being capable of binding in the vicinity of an antigen recognition region thereby reducing the fluctuation of the binding between a fluorescent labeling substance and a target substance.

A fluorescent labeling substance can be prepared by reacting and labeling a single domain antibody with a fluorescent dye. Generally, a functional group such as an amino group, a carboxyl group, a halogen or a nitro group is introduced in a fluorescent dye. Since a single domain antibody is a polypeptide, a reaction between a single domain antibody and a fluorescent dye can be carried out according to conditions well known to those skilled in the art. For example, a covalent bond can be formed when a functional group of a fluorescent dye is activated and mixed with a single domain antibody and reacted in a temperature range of 4 to 65° C. for several hours. Unreacted fluorescent dye can be purified by a conventional method after the reaction is completed. Since the single domain antibody has excellent heat resistance, the single domain antibody can react even at a high temperature, which allows preparation of a fluorescent labeling substance under various reaction conditions. Note that, if a single domain antibody is produced by genetic engineering, an amino acid residue having an amino group, a carboxyl group, a thiol group or the like capable of reacting with a fluorescent dye may be introduced at a position where the binding of the fluorescent dye is desired so that the fluorescent dye having a functional group corresponding to the amino acid residue may react with the amino acid residue. In this way, a fluorescent dye can bind to the vicinity of a variable region, N-terminal, C-terminal, as well as, —NH₂ group derived from arginine, asparagine, glutamine and lysine, —SH group derived from cysteine, or other arbitrary position of the single domain antibodies. Note that a single domain antibody and a fluorescent dye may be bound to each other via an arbitrary linker.

The number of binding of fluorescent dye molecules to one single domain antibody molecule can be arbitrarily selected. One or more fluorescent dye molecules per single domain antibody molecule are preferable, and two to five fluorescent dye molecules per single domain antibody molecule are more preferable. The average mass of a single domain antibody is 12 to 15 kDa, and binding five or more molecules may impair a binding property to a target substance.

The target substance that can be measured by the present disclosure is not particularly limited as long as a single domain antibody capable of reacting with at least a part of the target substance as an epitope can be prepared. For example, the preferred mass is 1.5×10³ to 1×10⁸ Da, more preferably 1×10⁵ to 1×10⁸ Da. In terms of size, the stalk diameter is preferably 1 nm to 10 μm, and more preferably 3 nm to 10 μm. Measurement can be performed within these ranges. In addition, when target substances are classified by their origins and characteristics, it is possible to measure biological substances, drugs, viruses, bacteria and the like. The biological substances include various components produced inside the bodies of organisms, various components excreted from living bodies, and organisms themselves where the organisms include both plants and animals. Further, the drugs are not limited to drugs to be administered to humans and animals, and include agrochemicals and the like.

The biological substances include: hormones, which are physiologically active substances synthesized and secreted by endocrine organs such as the hypothalamus, pituitary gland, thyroid gland, parathyroids, adrenal glands, pancreas, and gonads; metabolites such as nucleic acids, uric acids, purines, C-reactive proteins (CRP), apolipoproteins, HDLs, LDLs, and glycated hemoglobins; shellfish and bacterial toxins such as mycotoxin, aflatoxin B1 and botulinum toxin A; plant-derived alkaloids such as morphine, atropine, quinine, and cocaine; bacteria such as Escherichia coli, Streptococcus, Bacillus, Salmonella, and Pseudomonas aeruginosa. Further, the drugs include antibiotics such as chloramphenicol and cyclosporine and agrochemicals that are used for improving agricultural efficiency, such as bactericides, fungicides, insecticides, herbicides, rodenticides, and plant growth regulators. A virus is a microscopic infectious structure that replicates itself using the cells of other organisms. Measurable viruses include influenza viruses, corona viruses, hepatitis B viruses, hepatitis A viruses, hepatitis C viruses, and AIDS viruses.

The fluorescence polarization immunoassay according to the present disclosure measures the fluorescence polarization of a fluorescent labeling substance to which a target substance is bound. A sample is appropriately diluted with pure water or other diluting solution in accordance with the characteristics of the target substance contained in the sample, and, if necessary, impurities are removed to prepare a sample solution. This sample solution is mixed with the fluorescent labeling substance, thereby binding the target substance to the fluorescent labeling substance. Then, the fluorescence polarization of the conjugate of the target substance and the fluorescent labeling substance is measured. Any polarization measuring device can be used to measure the fluorescence polarization. The measurement may be carried out within a temperature range where the target substance is not denatured, that is, within a temperature range of 4 to 40° C., preferably at a constant temperature within the above range. Quantification of a target substance may be performed by preparing a calibration curve in advance through operations in the same manner as above using solutions containing the target substance at known concentrations and comparing the calibration curve with the measured value of the sample solution.

The same applies to when analyzing a microorganism such as a bacterium as a target substance. A single domain antibody that specifically binds to any part of a bacterium as an epitope is prepared in advance, and a fluorescent labeling substance in which this single domain antibody and a fluorescent dye are bound is prepared. The fluorescent labeling substance is added to a sample solution to bind bacteria contained in the sample solution to the fluorescent labeling substance. Then, a change in the fluorescence polarization of the fluorescent labeling substance to which the bacteria is bound may be measured. The amount of bacteria can be quantified by using a calibration curve prepared in advance using solutions containing the target substance at known concentrations and comparing the calibration curve with the measured value of the sample solution. The same applies when measuring a virus instead of a bacterium.

In the present disclosure, there is no limitation on the device as long as the fluorescence polarization can be measured. On the other hand, by using a measuring device comprising a microchannel, highly sensitive measurement can be performed with a trace amount of sample. The microchannel used in a fluorescence polarization measurement method must be made of a material that does not affect the fluorescence polarization, and, for this reason, PDMS is often used. However, it was found that when a fluorescent labeling substance formed by binding a Fab antibody to a fluorescent dye was used, the conjugate of the fluorescent labeling substance and the target substance adhered to PDMS. On the contrary, when a fluorescent labeling substance formed by reacting a single domain antibody with a fluorescent dye is used, the conjugate of the fluorescent labeling substance and the target substance does not adhere to the microchannel formed by PDMS, which enables quick and accurate measurement. Although number of variable region of a single domain antibody is one, a Fab antibody contains four variable regions, and correspondingly the volume of a Fab antibody is three- to four-fold. It is presumed that such a difference in volume and structure causes a difference in the adhesive ability to PDMS.

An example of the fluorescence polarization measuring device having such a microchannel is a fluorescence polarization measuring device used in Example 4 as will be described later. Although fluorescence polarization is measured by supplying a fluorescent labeling substance, a sample, a fluorescent labeling substance to which a target substance has already bound, or the like to the microchannel, it is preferable to use a microfluidic channel to form a sample illumination part where at least a fluorescent labeling substance emits fluorescence when irradiated with excitation light to measure fluorescence polarization. With the microchannel, a plurality of channels can be formed in the effective visual field of an optical observation part where fluorescence polarization is measured, and a plurality of samples can be simultaneously measured and image-analyzed. For example, even when the effective visual field of the optical observation part is about 3 mmφ, nine channels can be formed by setting each channel pitch to about 300 μm. The channel width and the inter-channel space can be arbitrarily set in accordance with this channel pitch. For example, if the channel width and the inter-channel space are equally spaced, the channel width can be set to 150 μm and the inter-channel space can be set to 150 μm. Since the measurement sensitivity increases as the channel depth is deeper, the channel depth can be set to 900 μm or the like. Note that, in order to increase the measurement sensitivity, the microchannel constituent material may be blackened. The aforementioned is only an example. There is a close relationship between the channel depth and channel width dimensions in terms of manufacturability, thus, for example, when the channel depth is 300 μm, the channel width can be 200 μm or more. When the channel width increases, the light extraction efficiency improves regardless of the configuration of the optical system, which improves measurement uniformity.

According to the fluorescence polarization measurement method of the present disclosure, the concentration of a target substance contained in a sample is measured in a range of 100 μM (pmol/L) to 10 μM (micromol/L), more preferably 1 to 1,000 nM (nanomol/L) regardless of whether or not a microchannel is used. Further, according to the fluorescence polarization measurement method of the present disclosure, the concentration of a target substance having a mass of 1.5×10³ to 1×10⁸ Da, more preferably 1×10⁵ to 1×10⁸ Da, can be quantified. Specifically, in the case of a target substance having a mass of about 150 kDa, the concentration can be measured in a range of 0.4 to 10,000 nM (nanomol/L). Moreover, as indicated in an example described later, by using a fluorescent labeling substance in which a single domain antibody is labeled with a fluorescent dye having the fluorescence life of 1 to 10 nanoseconds, even when a high molecular weight target substance such as IgG is to be quantified, the fluctuation range of the fluorescence polarization can be expanded as compared with the case of using a Fab antibody.

A second embodiment of the present disclosure is a fluorescent labeling substance in which a fluorescent dye is bound to a single domain antibody. As described above, the single domain antibody means an antibody composing by one variable region. A VHH antibody and a vNAR antibody can be used as the single domain antibody. A conventionally known Fab antibody or scFv antibody may be decomposed to extract one variable region that may be used as the single domain antibody. Further, the single domain antibody may be one that is produced by genetic engineering by referring to the DNA sequences of conventionally known VHH antibodies and vNAR antibodies or by using an antibody library or the like.

A fluorescent dye is a dye that emits fluorescence. The fluorescent dye preferably has a functional group capable of binding to a carboxyl group, an amino group, a hydroxyl group, a thiol, a phenyl group, or the like. This is because a single domain antibody often has a carboxyl group, an amino group, a hydroxyl group, a thiol, or a phenyl group, and if the fluorescent dye has a functional group capable of binding to any of the groups, it is easy to form a fluorescent labeling substance.

In addition, each fluorescent dye has its own fluorescence life. In the present disclosure, a fluorescent dye can be appropriately selected and used among a fluorescent dye having the fluorescence life of 1 to 10 nanoseconds, a fluorescent dye having the fluorescence life of more than 10 to 200 nanoseconds, and a fluorescent dye having the fluorescence life of more than 200 to 3,000 nanoseconds in accordance with the use purpose of the fluorescent labeling substance.

Examples of the fluorescent dye having the fluorescence life of 1 to 10 nanoseconds include indolenine, fluorescein compounds such as chlorotriazinylaminofluorescein, 4′-aminomethylfluorescein, 5-aminomethylfluorescein, 6-aminomethylfluorescein, 6-carboxyfluorescein, 5-carboxyfluorescein, 5- and 6-aminofluorescein, thioureafluorescein, and methoxytriazinylaminofluorescein, rhodamine derivatives such as rhodamine B, rhodamine 6G, and rhodamine 6GP; and, as registered trademark or product name, Alexa Fluor series such as Alexa Fluor 488, BODIPY series, DY series, ATTO series, Dy Light series, Oyster series, HiLyte Fluor series, Pacific Blue, Marina Blue, Acridine, Edans, Coumarin, DANSYL, FAN, Oregon Green, Rhodamine Green-X, NBD-X, TET, JOE, Yakima Yellow, VIC, HEX, R6G, Cy3, TAMRA, Rhodamine Red-X, Redmond Red, ROX, Cal Red, Texas Red, LC Red 640, Cy5, Cy5.5, and LC Red 705.

Examples of the fluorescent dye having the fluorescence life of more than 10 to 200 nanoseconds include naphthalene derivatives such as dialkylaminonaphthalenesulfonyl and pyrene derivatives such as N-(1-pyrenyl)maleimide, aminopyrene, pyrenebutanoic acid, and alkynylpyrene.

Examples of the fluorescent dye having the fluorescence life of more than 200 to 3,000 nanoseconds include metal complexes such as platinum, rhenium, ruthenium, osmium, and europium.

The fluorescent labeling substance of the present disclosure may be a fluorescent labeling substance in which a fluorescent dye having the fluorescence life of 1 to 10 nanoseconds is bound to a single domain antibody, a fluorescent labeling substance in which a fluorescent dye having the fluorescence life of more than 10 to 200 nanoseconds is bound to a single domain antibody, or a fluorescent labeling substance in which a fluorescent dye having the fluorescence life of more than 200 to 3,000 nanoseconds is bound to a single domain antibody. Using these fluorescent labeling substances, the concentration of a target substance having a mass within a range of 1.5×10³ to 1×10⁸ Da, more preferably 1×10⁵ to 1×10⁸ Da, can be measured by the fluorescence polarization immunoassay.

The binding between a fluorescent dye and a single domain antibody is preferably a covalent bond. A fluorescent labeling substance can be produced by reacting the above-described functional group of a fluorescent dye with a single domain antibody under conditions well known to those skilled in the art. For example, a covalent bond can be formed by activating the functional group of a fluorescent dye, mixing the fluorescent dye with a single domain antibody and reacting the mixture in a temperature range of 4 to 65° C. for several hours. After completion of the reaction, the unreacted fluorescent dye is removed by a conventional method. In addition, when producing a single domain antibody by genetic engineering, an amino acid residue having an amino group, a carboxyl group, a thiol group or the like capable of reacting with a fluorescent dye may be introduced at a position where the binding of the fluorescent dye is desired so that the fluorescent dye having a functional group corresponding to the amino acid residue may react with the amino acid residue. In this way, a fluorescent dye can bind to the vicinity of the variable region, N-terminal, C-terminal, —NH₂ group derived from arginine, asparagine, glutamine and lysine of other single domain antibodies, —SH group derived from cysteine, and other arbitrary position. Further, a single domain antibody and a fluorescent dye may be bound to each other via a linker. Examples of such a linker include an oligoethylene glycol and an alkyl chain. As indicated in an example described later, a fluorescent labeling substance in which a fluorescent dye is bound to an N-terminal corresponding to the vicinity of the variable region of a single domain antibody is excellent in sensitivity of fluorescence polarization.

The fluorescent labeling substance of the present disclosure can be used in fluorescence polarization immunoassay, sandwich immunoassay, and immunostaining.

The single domain antibody has a lower molecular weight than a Fab antibody and a scFv antibody, which are known as low molecular weight antibodies, easily rewinds to the natural structure even in a solution of a denaturing agent such as guanidine hydrochloride or urea or under a denaturing condition such as high temperature or high pressure, and has excellent heat resistance, pressure resistance, and chemical resistance. Particularly, such a single domain antibody has excellent heat resistance, in which the single domain antibody exhibits the same antigen-binding activity as before heating when returned to room temperature from a high temperature condition of 90° C. The single domain antibody is resistant to temperature changes and the like, which is advantageous for distribution, storage and the like. Furthermore, since the single domain antibody has high solubility in an aqueous solvent and is excellent in stability to a surfactant, the single domain antibody is also excellent in operability when the single domain antibody capable of specifically binding to a target substance is prepared by genetic engineering. In addition, when the single domain antibody is used in a fluorescence polarization immunoassay, a high molecular weight target substance can be measured using a highly versatile fluorescent dye with the fluorescence life of 1 to 10 nanoseconds and a sample can be measured even at low concentration, which is advantages, for example, in reducing the amount of sample.

### EXAMPLES

The following will describe the present disclosure with reference to examples. However, these examples are not intended to limit the present disclosure in any way.

### Example 1

(1) In order to quantify rabbit IgG, rabbit IgG (manufactured by Sigma-Aldrich Co. LLC) was dissolved in a phosphate buffered saline (PBS) (manufactured by FUJIFILM Wako Pure Chemical Corporation) and 9-level rabbit IgG solutions, 3,230 nM (nanomol/L), 1,080 nM, 360 nM, 120 nM, 40 nM, 13 nM, 4.4 nM, 1.5 nM, and 0.49 nM, were prepared.

(2) Anti-Rabbit IgG Alpaca-mono, recombinant VHH Alexa Fluor 488 modified (VHH) (manufactured by ChromoTek GmbH, mass 15 kDa) was used as a fluorescent labeling substance in which a single domain antibody having a binding ability to a target substance (rabbit IgG) is labeled with a fluorescent dye. The fluorescent labeling substance was diluted 100-fold with PBS to prepare a fluorescent labeling substance solution.

(3) Fetal bovine serum (FBS) (manufactured by Biowest) was diluted 10-fold with PBS to obtain an FBS solution.

(4) The fluorescent labeling substance solution 16 μL (containing 5 μg/mL of VHH), 60 μL of FBS solution, 60 μL of 9-level rabbit IgG solutions, and 464 μL of PBS were mixed to prepare nine kinds of 600 samples for creating a standard curve. By this preparation, the antibody concentration became 10 nM (nanomol/L). After preparing the samples, the fluorescence polarization of each sample was measured after standing in the dark for two hours at room temperature.

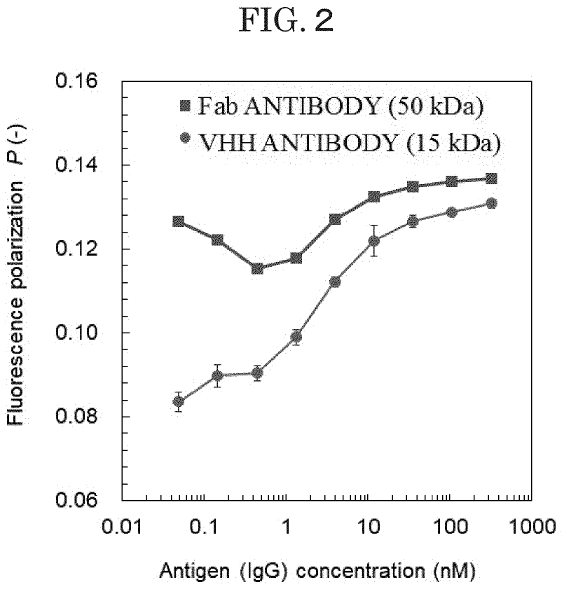

(5) As the fluorescence polarization measuring device, a spectrofluorometer F7100 (manufactured by Hitachi High-Tech Science Corporation) was used for measurement in the fluorescence polarization mode. The excitation wavelength was set to 490 nm; the detection wavelength, 510-540 nm; the scan speed, 60 nm/min; the initial waiting time, 0 s; the fluorescence side slit, 10 nm; the excitation side slit, 10 nm; and the response, 0.002 s. A sample of 150 μL containing 0.049 nM (nanomol/L) rabbit IgG for preparing a standard curve was placed in a cell and G value was measured. The sample for preparing a standard curve was placed in a quartz cell and the fluorescence polarization was measured. All data were measured 3 times. The results are illustrated in FIG. 2 as the VHH antibody (15 kDa).

### Comparative Example 1

Operations were conducted in the same way as Example 1 except that Anti-Rabbit IgG Alexa labeled Fab fragment (Fab) (manufactured by Jackson ImmunoResearch Inc. mass 50 kDa) was used instead of Anti-Rabbit IgG Alpaca-mono, recombinant VHH Alexa Fluor 488 modified (VHH). The results are illustrated in FIG. 2 as the Fab antibody (50 kDa).

As illustrated in FIG. 2, the fluorescence polarization of Comparative Example 1 using the Fab antibody fluctuated in a range of 0.108 to 0.138, and the fluctuation range was 0.03. On the other hand, the fluorescence polarization of Example 1 using the VHH antibody fluctuated in a range of 0.082 to 0.13, and the fluctuation range was 0.048. By using the VHH antibody, the fluctuation range was expanded by 1.6 times as compared with the case of using the Fab antibody.

Further, the results of Example 1 and Comparative Example 1 were used to create a sigmoid curve under the conditions illustrated in Table 1, and the lower limit of quantification and the upper limit of quantification were calculated.

TABLE 1

| | |
|---|---|
| lower limit of | concentration when Plow = Pmin + 3SDlow |
| quantification | |
| upper limit of | concentration when Phigh = Pmax − 3SDhigh |
| quantification | |
| Pmin | m₁ of sigmoid curve |
| Pmax | m₂ of sigmoid curve |
| SDlow | standard deviation at the minimum concentration (0.049 |
| | nM (nanomol/L)) |
| SDhigh | standard deviation at the maximum concentration (323 |
| | nM (nanomol/L)) |
| sigmoid curve | P = m₁ + (m₂ − m₁)/(1 + (C/m₃)^{m4}) |
| P | standard deviation at the minimum concentration (0.049 |
| | nM (nanomol/L)) |
| C | standard deviation at the maximum concentration (323 |
| | nM (nanomol/L)) |
| m₁, m₂, m₃, m₄ | fitting parameters (m₄ is a negative value) |

As the result, the lower limit of quantification of Comparative Example 1 using the Fab antibody was calculated as 5.4 nM (nanomol/L) and the upper limit of quantification was 18 nM (nanomol/L), while the lower limit of quantification of Example 1 using the VHH antibody was calculated as 0.45 nM (nanomol/L) and the upper limit of quantification was 41 nM (nanomol/L). It was found even a concentration as low as 0.45 nM (nanomol/L) was measurable.

### Example 2

(1) Anti-Her2 Alpaca, monoclonal, recombinant VHH (manufactured by QVQ Holding BV; 1 mg/mL) was used as a single domain antibody having a binding ability to a target substance Her2 (manufactured by R&D Systems, Inc. ErbB2/Her2 Fc chimeric recombinant protein).

(2) This antibody 20 μL and 2.75 equivalents of tris(2-carboxyethyl) phosphine hydrochloride (TCEP-HCl) aqueous solution (manufactured by FUJIFILM Wako Pure Chemical Corporation) were mixed and allowed to stand at 37° C. in the dark for two hours. Six equivalents of Alexa Fluor 488 maleimide (manufactured by Thermo Scientific Inc.) was added to this solution, and the mixture was allowed to stand in the dark for two hours at room temperature, and then purified twice using a Zeba column (7 kDa) to produce a fluorescent labeling substance. In this fluorescent labeling substance, Alexa Fluor 488 was bound to the SH group of the antibody.

(3) The target substance Her2 was diluted 50-fold with a phosphate buffered saline (PBS) (manufactured by FUJIFILM Wako Pure Chemical Corporation). This diluted solution was further serially diluted 5-fold with PBS to prepare Her2 solutions of 6 levels of concentrations, 410 nM (nanomol/L), 82 nM, 16 nM, 3.3 nM, 0.66 nM and 0.13 nM.

(4) A solution 92.5 μL obtained by diluting the fluorescent labeling substance solution with PBS to the concentration of 44 nM (nanomol/L), 200 μL of the Her2 solution of each concentration, 5 μL of fetal bovine serum (FBS) (manufactured by Biowest), and 402.5 μL of PBS were mixed to obtain six kinds of 500 μL samples for creating a standard curve. By this preparation, the VHH in the samples became 8.2 nM (nanomol/L). In addition, 80 μL of the fluorescent labeling substance solution, 2 of fetal bovine serum (FBS) (manufactured by Biowest), and 161 μL of PBS were mixed to obtain a total of 200 μL of control sample not containing Her2. After preparing each sample, the sample was allowed to stand in the dark for two hours at room temperature, then, the fluorescence polarization was measured.

(5) Tecan Infinite 200 PRO F Plex was used as the fluorescence polarization measuring device. The excitation wavelength was set to 490 nm, and the detection wavelength, 510 to 540 nm.

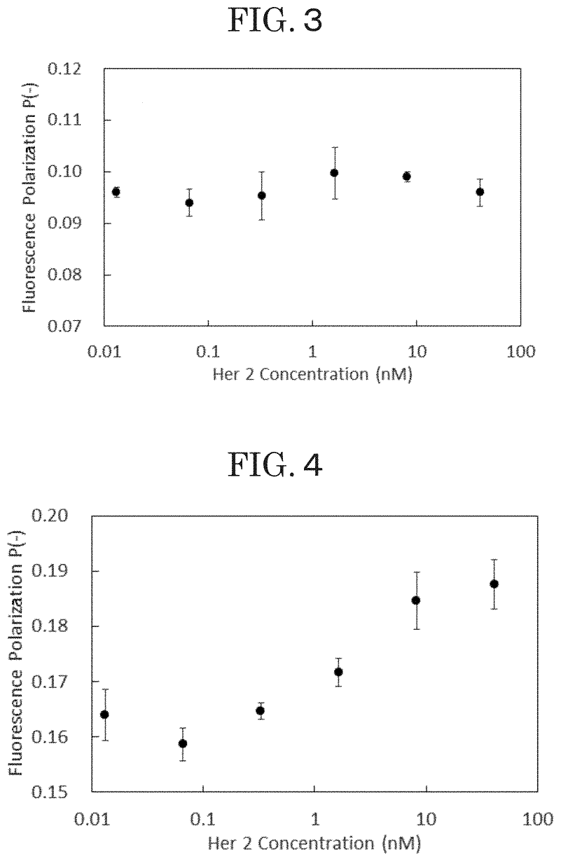

The results are illustrated in FIG. 3. It was possible to confirm an increase in the fluorescence polarization according to the Her2 concentration where the concentration was in a range of 0.66 to 1.6 nM (nanomol/L).

### Example 3

(1) Anti-Her2 Alpaca, monoclonal, recombinant VHH (manufactured by QVQ Holding BV; 1 mg/mL) was used as a single domain antibody having a binding ability to a target substance Her2 (manufactured by R&D Systems, Inc. ErbB2/Her2 Fc chimeric recombinant protein). This antibody 20 μL and 5 equivalents of Alexa Fluor 488 SDP ester (manufactured by Thermo Scientific Inc.) were mixed under the condition of pH 8.5, and the mixture was stirred in the dark at room temperature for 1 hour. Then, the mixture was purified twice using a Zeba column (7 kDa) to prepare a fluorescent labeling substance. In this fluorescent labeling substance, the fluorescent dye Alexa Fluor 488 was bound to the —NH₂ group of the antibody.

(2) The fluorescence polarization was measured by the same operations as in Example 2 except that the fluorescent labeling substance obtained in (1) above was used. The results are illustrated in FIG. 4. When the VHH antibody, to which —NH₂ group Alexa Fluor 488 was bound, was used, it was possible to confirm an increase in the fluorescence polarization according to the Her2 concentration where the concentration was in a range of 0.66 to 8.2 nM (nanomol/L). In addition, the fluorescence polarization fluctuated between 0.158 and 0.187, and it was possible to confirm that the fluorescence polarization was more sensitive and fluctuated according to the concentration than Example 2 where Alexa Fluor 488 was bound to the —SH group of the single domain antibody.

### Example 4

(1) To quantify rabbit IgG, rabbit IgG (manufactured by Sigma-Aldrich Co. LLC) and a phosphate buffered saline (PBS) (manufactured by FUJIFILM Wako Pure Chemical Corporation) were used to prepare 7-level rabbit IgG solutions of 1,080 nM (nanomol/L), 360 nM, 120 nM, 40 nM, 13 nM, 4.4 nM and 0.15 nM.

(2) Anti-Rabbit IgG Alpaca-mono, recombinant VHH Alexa Fluor 488 modified (VHH) (manufactured by Chromotek GmbH, mass 15 kDa) was used as a fluorescent labeling substance in which a single domain antibody having a binding ability to a target substance (rabbit IgG) was labeled with a fluorescent dye. This fluorescent labeling substance was diluted 100-fold with PBS to prepare a fluorescent labeling substance solution.

(3) Bovine serum albumin (BSA) (manufactured by Abcam Inc.) was dissolved in PBS to obtain a 1% BSA solution.

(4) Fetal bovine serum (FBS) (manufactured by Biowest) was diluted 10-fold with PBS to obtain an FBS solution.

(5) The fluorescent labeling substance solution 8 μL (containing 5 μg/mL of VHH), 30 μL of the BSA solution, 30 μL of the rabbit IgG solution of each concentration, and 232 μL of PBS were mixed to obtain 7-level 300 μL samples for preparing a standard curve.

(6) In addition, 30 μL of rabbit IgG solutions respectively containing 13 nM and 120 nM rabbit IgG were separately prepared, and 8 μL of the fluorescent labeling substance solution (containing 10 nM of VHH), 30 μL of the FBS solution, 30 μL of the rabbit IgG solution, and 232 μL of PBS were mixed to prepare a sample for measurement.

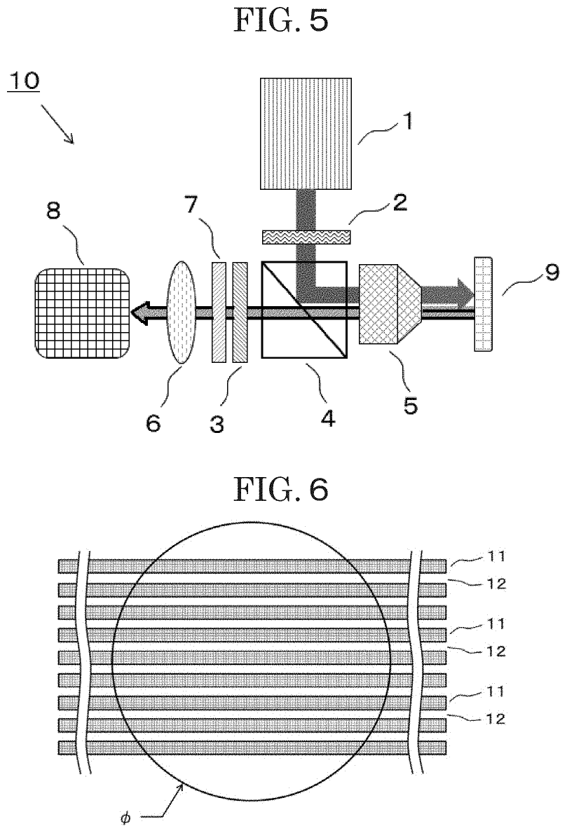

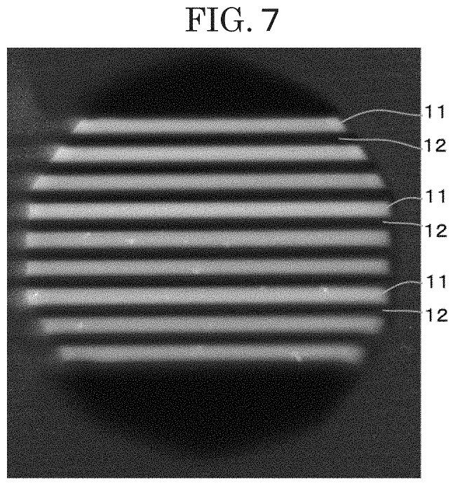

(7) Using a fluorescence polarization measuring device having nine microchannels, the fluorescence polarization of the samples for preparing a standard curve and the samples for measurement were measured with the excitation wavelength of 470±5 nm and the detection wavelength of 520±5 nm. Note that, of the nine microchannels, seven microchannels were injected with the samples of seven levels for creating a standard curve described in (4) above, the remaining two microchannels were injected with the samples for measurement of two levels described in (5) above, and all the samples were measured at the same time. FIG. 5 illustrates a schematic configuration of the fluorescence polarization measuring device used. The device 10 mainly includes an LED light source 1, an excitation filter 2, a fluorescence filter 3, a dichroic filter 4, an objective lens 5, an imaging lens 6, a liquid crystal element 7, a digital imaging device (a CMOS or CCD camera) 8, and a sample illumination part 9. The excitation light from the LED light source 1 having a central wavelength of 470 nm is irradiated to the samples in the sample illumination part 9 via the excitation filter 2 and the objective lens 5, and the fluorescence emitted by the sample is transmitted through the dichroic filter 4 and the fluorescence filter 3, then, the transmitted light is acquired by the CMOS camera 8. When voltage is applied to the liquid crystal element 7 arranged between the fluorescence filter 3 and the imaging lens 6 and modulated, the polarization direction of the transmitted fluorescence can be modulated. The modulation frequency and the captured frequency at the CMOS camera 8 are synchronized to acquire and calculate an image, and a polarization degree P is calculated as the two-dimensional image. The effective visual field of the optical observation part of the sample illumination part 9 of this device 10 is about 3 mmφ. The nine channels are used to simultaneously measure the samples for a standard curve and the samples for measurement. As illustrated in FIG. 6, the channel width 11 and the inter-channel space 12 are equally spaced with the channel width of 150 μm and the inter-channel space of 150 μm within the effective visual field of φ3 mm illustrated as a circle. Note that the channel depth is 900 μm. By forming a plurality of microchannels in the sample illumination part 9, a plurality of samples can be measured at the same time. FIG. 7 illustrates an image of the measured fluorescence polarization of the microchannels used in Example 4.

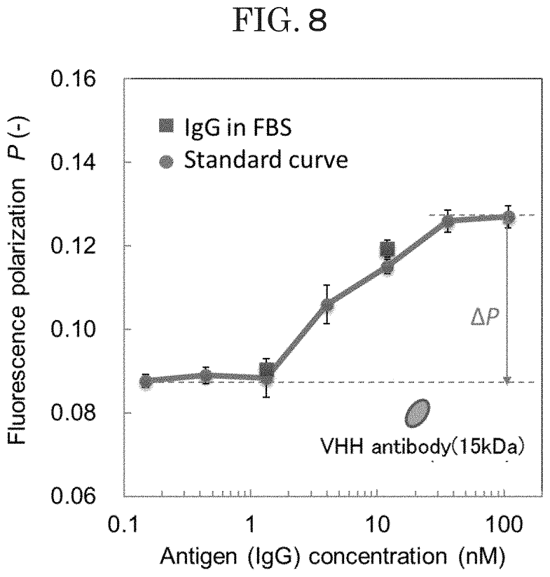

(8) The results are illustrated in FIG. 8. In FIG. 8, black circles represent a standard curve, and black squares are the measurement results of the samples containing 1.3 nM (nanomol/L) and 12 nM (nanomol/L) of IgG. Using the VHH antibody, measurement was possible even with a measuring instrument equipped with a microchannel.

### Example 5

(1) In order to quantify rabbit IgG, rabbit IgG (manufactured by Sigma Aldrich Co. LLC) was dissolved in a phosphate buffered saline (PBS) (manufactured by FUJIFILM Wako Pure Chemical Corporation) to prepare 9-level rabbit IgG solutions 3,280 nM (nanomol/L), 1,080 nM, 360 nM, 120 nM, 40 nM, 13 nM, 4.4 nM, 1.5 nM, and 0.49 nM.

(2) Anti-Rabbit IgG Alpaca-mono, recombinant VHH Alexa Fluor 488 modified (VHH) (Chromotek GmbH, mass 15 kDa), as a fluorescent labeling substance in which a single domain antibody having a binding ability to a target substance (rabbit IgG) was labeled with a fluorescent dye, was diluted 1000-fold with PBS to prepare a fluorescent labeling substance solution.

(3) The fluorescent labeling substance solution 2.7 μL (containing 0.5 μg/mL of VHH), 10 μL of the rabbit IgG solution of each concentration, and 87.3 μL of PBS were mixed to obtain 9-level 100 μL samples for preparing a standard curve.

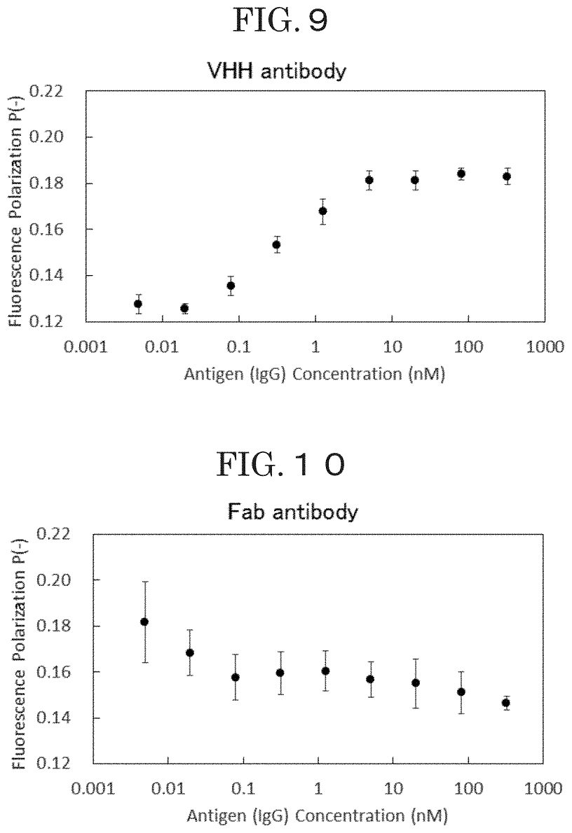

(4) The fluorescence polarization of the samples for creating a standard curve were measured using the fluorescence polarization measuring device having nine microchannels with the excitation wavelength of 470±5 nm and the detection wavelength of 520±5 nm. Note that the nine microchannels were injected with the nine levels of samples for creating a standard curve described in (4) above and measured at the same time. The fluorescence polarization measuring device used was the same as the one in Example 4. FIG. 9 illustrates the measurement results of Example 5.

### Comparative Example 2

Operations were conducted in the same way as Example 5 except that Anti-Rabbit IgG Alexa labeled Fab fragment (Fab) (manufactured by Jackson ImmunoResearch Inc., mass 50 kDa) was used instead of Anti-Rabbit IgG Alpaca-mono, recombinant VHH Alexa Fluor 488 modified (VHH). FIG. 10 illustrates the measurement results of Comparable Example 2.

(Results)

As illustrated in the results of Example 5 in FIG. 9, the measuring device having microchannels was able to easily quantify the amount of IgG using a VHH antibody. On the other hand, as indicated in FIG. 10, when a Fab antibody was used with the measuring device having microchannels, the increase in the fluorescence polarization according to the concentration as observed in Example 1 was not observed, and it was difficult to quantify the amount of IgG. This is presumed to be because the Fab antibody has a larger molecular weight than the VHH antibody and is easily adsorbed on the wall surface of the microchannel. Like the Fab antibody, when an antibody is adsorbed, the rotational diffusion of the antibody molecules is suppressed, thus, the antibody exhibits a high fluorescence polarization value even when the antibody has not reacted with an antigen. As such, it is considered that the measuring device having microchannels could not obtain the result of an increased fluorescence polarization value according to the antigen concentration as in Example 1, and the measurement of the antigen became difficult. On the other hand, like the VHH antibody, when an antibody is hardly adsorbed on the channel, the fluorescence polarization value increases as the antigen concentration increases even with a measuring device having microchannels, which enabled measurement with high measurement sensitivity as in Example 1.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A fluorescence polarization immunoassay for analyzing a target substance contained in a sample, the fluorescence polarization immunoassay comprising:
a binding step for binding a fluorescent labeling substance, in which a single domain antibody having a binding ability to the target substance is labeled with a fluorescent dye, to the target substance contained in the sample; and
a measuring step for measuring a change in fluorescence polarization of the fluorescent labeling substance to which the target substance is bound.

2. The fluorescence polarization immunoassay according to claim 1, wherein the single domain antibody is a VHH antibody or a vNAR antibody.

3. The fluorescence polarization immunoassay according to claim 1, wherein the fluorescent dye is one or more selected from a group consisting of fluorescein, dansyl, pyrene, rhodamine, dialkylaminonaphthalene, dialkylaminonaphthalenesulfonyl, indorenine, and ruthenium.

4. The fluorescence polarization immunoassay according to claim 1, wherein the fluorescent dye has a fluorescence life of 1 to 3,000 nanoseconds.

5. The fluorescence polarization immunoassay according to claim 1, wherein the fluorescent labeling substance, the sample, or the fluorescent labeling substance to which the target substance is bound, is supplied to a microchannel to measure fluorescence polarization.

6. The fluorescence polarization immunoassay according to claim 1, wherein the target substance is a biological substance, a drug, or a virus.

7. A fluorescent labeling substance in which a fluorescent dye is bound to a single domain antibody.
